(19) United States
(12) Patent Application Publication
(10) Pub. No.: US 2013/0030478 A1
(43) Pub. Date: Jan. 31, 2013

(54) ANCHOR WIRE SYSTEM AND METHOD

(71) Applicant: Jose A. Rodriguez, Miami, FL (US)

(72) Inventor: Jose A. Rodriguez, Miami, FL (US)

(21) Appl. No.: 13/557,482

(22) Filed: Jul. 25, 2012

Related U.S. Application Data
(60) Provisional application No. 61/511,632, filed on Jul. 26, 2011.

(51) Int. Cl. A61B 17/84

(52) U.S. Cl. 606/323

(57) ABSTRACT

A medical guide wire comprising a guide wire hollowed sleeve member and a retention member provided through a hollowed central portion of the sleeve member. The sleeve member includes at least one retention member operational port allowing the retention arm to laterally extend therethrough and retract therein as desired. The functional end of the locking member can be of any reasonable shape and may include one or multiple extending retention arms. A solid penetrating head is integrated into the hollowed sleeve member or the retention member allowing for direct insertion during use. The operational port may be located through either a sidewall or the distal end of the sleeve member. The medical guide wire can be functionally reversed using a feature of a central actuating member to retain the retention arms of a respective hollowed sleeve member in a retracted, insertion configuration and release the retention arms for deployment.

### CROSS-REFERENCE TO RELATED APPLICATION

This Non-Provisional Utility application claims the benefit of co-pending U.S. Provisional Patent Application Ser. No. 61/511,632, filed on Jul. 26, 2011, which is incorporated herein in its entirety.

### FIELD OF THE INVENTION

The present invention relates to an improved surgical guide wire for use in surgical procedures such as orthopedic procedures, more so, providing a guide wire having an extendable and retractable anchor feature provided proximate a distal end thereof.

### BACKGROUND OF THE INVENTION

In certain surgical procedures, a guide wire is used in combination with a surgical tool such as a Jamshidi needle. The Jamshidi needle is used to form a hole through bone as a first step in certain medical procedures like attaching a screw to a pedicle. The guide wire is inserted through the Jamshidi needle into the bone. The guide wire is used as a portal for certain surgical steps like guiding a tap, screw or screwdriver to the surgical site. The procedures oftentimes require the use of force, which can cause a properly positioned guide wire to migrate. This undesired movement of the guide wire may contact portions of the patient's anatomy and cause severe injury.

A guide wire is generally cylindrical, making it easy to move during use; in fact, the guide wire is designed to move during its installation, but once installed known guide wires may continue to move requiring extreme care during use. The cross sectional size of the guide wire is limited by the corresponding size of the tools and devices it is used with. Each tool or device is provided with a through bore for receiving the guide wire, limiting the size and type of wire that can be used. Additionally, the guide wire is typically removed by passing through a through bore in a device or tool. Thus, to date, only guide wires with a small diameter, generally cylindrical round cross section, and have been used which presents the problem in their use.

To prevent undesired translation of the guide wire several attempts have been made to anchor the inserted end subsequent to proper positioning. In one known solution discloses a cross pin and setscrew femoral and tibial fixation device for mounting a ligament graft. The device includes a drill guide for drilling a transverse hole. The drill guide is releasable from a first twist drill so as to leave it in place. The first twist drill is used to guide further drilling and passage of a fastener device. A guide wire or the first twist drill is used for guiding a second twist drill for enlarging the transverse hole and for guiding and turning a cannulated fastener device into a femoral bone end of the ligament graft. Another known solution discloses a surgical drill guide including a handle and an arm having an end that contacts a bone. The handle includes a plurality of slots or channels, which receive a sleeve. The sleeve is used to guide a guide wire into the bone. The guide wire serves as a guide for drilling a tunnel into the bone. While these known solutions are somewhat useful, there is no feature on the guide wire to limit the extent of its insertion subsequent to it passing through the bone.

In another known solution, there is disclosed various devices and system for placing bone stabilization components in an individual. In particular, the bone stabilization components are placed on the spine. Various tools, including a guide wire, are employed to properly locate, place and secure the devices in an individual. There is disclosed in another known solution a device for accessing the pedicle of a vertebra including a Jamshidi needle. This known solution uses a specially configured targeting needle having a sharpened point for accurately placing the targeting needle and a positioning needle with respect to a pedicle of a patient. However, these known solutions do not provide any features to limit the undesired forward translation of a guide wire subsequent to positioning within a patient.

Further, there is a known solution that discloses a guide wire having a deformable tip that increases the cross-sectional area of the tip once properly positioned. The tip is generally deformed at a right angle with respect to the longitudinal axis of the guide wire. The tip is deformed by employing a thermally deformable material to fabricate the guide wire. As the temperature of the guide wire is changed the tip then expands/contracts accordingly. This known solution while somewhat useful has substantial drawbacks. First, to deflect the tip of the guide wire at a right angle (90 degrees) takes significantly more volumetric space than the space pierced by a positioning needle such as a Jamshidi needle. As the tip deflects through the 90 degrees, it is possible to damage adjacent anatomical structures within the surgical site. Second, this use of this known solution requires that a secondary needle be used in combination with the Jamshidi needle, where the secondary needle provides a sharpened point to facilitate proper placement and penetration of the desired bone structure. Finally, this known solution requires significant volumetric space to deploy the deformable tip of the guide wire.

Efforts to provide an anchor wire that overcomes the drawbacks in the prior art have not met with significant success to date. As a result, there is a need in the art for an anchor wire (guide wire) that provides increased functionality with multiple medical treatments prevents undesired forward translation of the wire and is usable with traditional surgical tools and devices. It is further desired to provide a guide wire that enables resistance to prevent accidental pullout during use.

### SUMMARY OF THE INVENTION

The basic inventive concept provides an anchoring guide wire assembly for use during a surgical procedure where the anchor guide wire includes a feature to temporarily secure the wire assembly in location.

A first aspect of the present invention provides an anchoring guide wire assembly for use in a surgical procedure, the anchoring guide wire assembly comprising:

a guide wire hollowed sleeve member having a guide wire hollowed sleeve member distal end located at a first end and a guide wire hollowed sleeve member proximal end located at an opposite end, at least one retention member operational port formed adjacent to a guide wire penetrating head formed on the guide wire distal end, a central aperture formed extending longitudinally through the guide wire hollowed sleeve member between the guide wire hollowed sleeve member proximal end and the at least one retention member operational port, and a solid penetrating head formed at the guide wire hollowed sleeve member distal end; and

a guide wire location retention member having a retention member distal end, a retention member proximal end and a retention member shank section spanning therebetween, the distal end configured with at least one extending retention arm,

wherein the guide wire location retention member is slideably assembled through the central aperture, the at least one extending retention arm is configured to extend through the at least one retention member operational port such that when the at least one extending retention arm is in a deployed configuration, the retention member extends laterally outward from the distal end of the guide wire hollowed sleeve member.

A second aspect of the present invention, the anchoring guide wire assembly is fabricated of medical grade materials, sterilized, and packaged to retain said sterility.

In another aspect, the guide wire hollowed sleeve member is fabricated having a single retention member operational port piercing through a sidewall and the guide wire location retention member is fabricated having a single extending retention arm formed to bend laterally from a longitudinal axis of the guide wire location retention member when the extending retention arm is discharged through the single retention member operational port.

In yet another aspect, the guide wire hollowed sleeve member is fabricated having two retention member operational ports piercing through a sidewall and the guide wire location retention member is fabricated having two extending retention arms joined together at a location proximate said guide wire location retention member proximal end.

A second embodiment of the present invention provides an anchoring guide wire assembly for use in a surgical procedure, the anchoring guide wire assembly comprising:

a guide wire hollowed sleeve member having a guide wire hollowed sleeve member proximal end located at a first end and a guide wire hollowed sleeve member distal end located at an opposite end, a central aperture formed extending longitudinally through the guide wire hollowed sleeve member between the guide wire hollowed sleeve member proximal end and the guide wire hollowed sleeve member distal end, and a retention member operational port formed at the guide wire hollowed sleeve member distal end; and

a guide wire location retention member having a retention member proximal end, a retention member distal end and a retention member shank section spanning therebetween, a solid guide wire assembly penetrating head carried at the retention member distal end, and at least one extending retention arm formed to extend laterally from the retention member shank section when placed into a natural state,

wherein the guide wire location retention member is slideably assembled through the retention member operational port, the retention member is retracted within the central aperture when the anchoring guide wire assembly is placed into a retracted, installing configuration and the retention member is extended laterally outward from the distal end of the guide wire hollowed sleeve member when the anchoring guide wire assembly is placed into a deployed configuration.

In yet another aspect, the guide wire location retention member is fabricated having a single extending retention arm formed to bend laterally from a longitudinal axis of the guide wire location retention member when the anchoring guide wire assembly is placed into a deployed configuration.

In yet another aspect, the guide wire location retention member is fabricated having at least two extending retention arms formed to bend laterally from a longitudinal axis of the guide wire location retention member when the anchoring guide wire assembly is placed into a deployed configuration. The at least two extending retention arms are preferably provided at equidistant spacing about a circumference of the anchoring guide wire.

A second embodiment of the present invention provides an anchoring guide wire assembly for use in a surgical procedure, the anchoring guide wire assembly comprising:

a guide wire hollowed sleeve member comprising:
a sleeve tubular segment,
a sleeve distal retention section comprising at least one extending retention arm, wherein each of said at least one extending retention arm is formed to extend laterally from a longitudinal axis of said sleeve tubular segment when placed into a natural state, each of said at least one extending retention arm having a retention arm distal end, and
a sleeve central aperture extending longitudinally through said sleeve tubular segment;

a central actuating member comprising:
an actuating member shaft having first end defined as an actuating member shaft proximal end and a second, opposite end defined as an actuating member shaft distal end, and
a solid penetrating head carried by said actuating member shaft distal end, said solid penetrating head shaped to include a retention arm distal end retention feature, wherein said retention arm distal end retention feature is defined to releasably engage with each of said retention arm distal end to temporarily retain said each of said at least one extending retention arm in a retracted configuration;

wherein said central actuating member is slideably assembled through said sleeve central aperture, said retention member is retracted within said central aperture when said anchoring guide wire assembly is placed into a retracted and each of said retention arm distal ends is retained in a retracted configuration by said retention arm distal end retention feature when said anchoring guide wire assembly is placed into an insertion configuration and said each of said retention arm distal ends is released from said retention arm distal end retention feature, enabling each of said at least one first retention arm to deploy when said anchoring guide wire assembly is placed into a deployed configuration.

These and other advantages of the invention will be further understood and appreciated by those skilled in the art by reference to the following written specification, claims and appended drawings.

### BRIEF DESCRIPTION OF THE DRAWINGS

The components in the figures are not necessarily to scale, emphasis instead being placed upon illustrating the principles of the invention. The invention will now be described, by way of example, with reference to the accompanying drawings, in which:

FIG. 1 presents a section view of a Vertebra including a Jamshidi Needle having a guide wire passing therethrough and exhibiting an extended anchor feature, the needle being shown extending through a pedicle;

FIG. 2 presents a sectioned side view of a first exemplary reformable functioning end of the anchor wire illustrated in a retracted state;

FIG. 3 presents a sectioned side view of the first exemplary reformable functioning end of the anchor wire originally presented in FIG. 2, the exemplary embodiment illustrated in a deployed anchor state;

FIG. 4 presents a magnified side view of the first exemplary reformable functioning end of the anchor wire originally presented in FIG. 3;

FIG. 5 presents a front view of a first exemplary reformable functioning end of the anchor wire in a deployed anchor state and attached to a pelvic bone;

FIG. 6 presents a front view of a first exemplary reformable functioning end of the anchor wire in a deployed anchor state and attached to an upper end of a humerus bone;

FIG. 7 presents a side view of an exemplary position engaging mechanism of the anchor wire, the anchor wire being illustrated in a retracted state;

FIG. 8 presents a side view of the exemplary position retaining mechanism of the anchor wire originally presented in FIG. 7, the anchor wire being illustrated in a deployed anchor state;

FIG. 9 presents a side view of a second exemplary reformable functioning end of the anchor wire having a single anchor arm, the reformable functioning end illustrated in a retracted state;

FIG. 10 presents a side view of the second exemplary reformable functioning end of the anchor wire originally presented in FIG. 13, the exemplary embodiment illustrated in a deployed anchor state;

FIG. 11 presents a side view of a second exemplary reformable functioning end of the anchor wire having a retention member, the reformable functioning end illustrated in a retracted state;

FIG. 12 presents a side view of the second exemplary reformable functioning end of the anchor wire originally presented in FIG. 12, the exemplary embodiment illustrated in a deployed anchor state;

FIG. 13 presents a side view of a third exemplary reformable functioning end of the anchor wire having a pair of extending retention arms, the reformable functioning end illustrated in a retracted state;

FIG. 14 presents a side view of the third exemplary reformable functioning end of the anchor wire originally presented in FIG. 13, the exemplary embodiment illustrated in a deployed anchor state;

FIG. 15 presents a side view of a third exemplary reformable functioning end of the anchor wire having multiple extending retention arms, the reformable functioning end illustrated in a retracted state;

FIG. 16 presents a side view of the third exemplary reformable functioning end of the anchor wire originally presented in FIG. 15, the exemplary embodiment illustrated in an extended anchor state;

FIG. 17 presents a side view of a fourth exemplary reformable functioning end of the anchor wire having a single retention ember and a broadened head configuration, the reformable functioning end illustrated in a retracted state;

FIG. 18 presents a side view of the fourth exemplary reformable functioning end of the anchor wire originally presented in FIG. 17, the exemplary embodiment illustrated in a deployed anchor state;

FIG. 19 presents an isometric view of a fifth exemplary reformable functioning end of the anchor wire having a single retention ember and a broadened head configuration, the reformable functioning end illustrated in a retracted state;

FIG. 20 presents an isometric view of the fifth exemplary reformable functioning end of the anchor wire originally presented in FIG. 19, the exemplary embodiment illustrated in a deployed anchor state;

FIG. 21 presents an isometric view of an exemplary reformable functioning outer sheath retained by a central sliding retention member, the reformable functioning end illustrated in a retracted state; and

FIG. 22 presents an isometric view of an exemplary reformable functioning outer sheath retained by a central sliding retention member, the exemplary embodiment illustrated in a deployed anchor state.

In the figures, like reference numerals designate corresponding elements throughout the different views of the drawings.

### DETAILED DESCRIPTION OF THE PREFERRED EMBODIMENTS

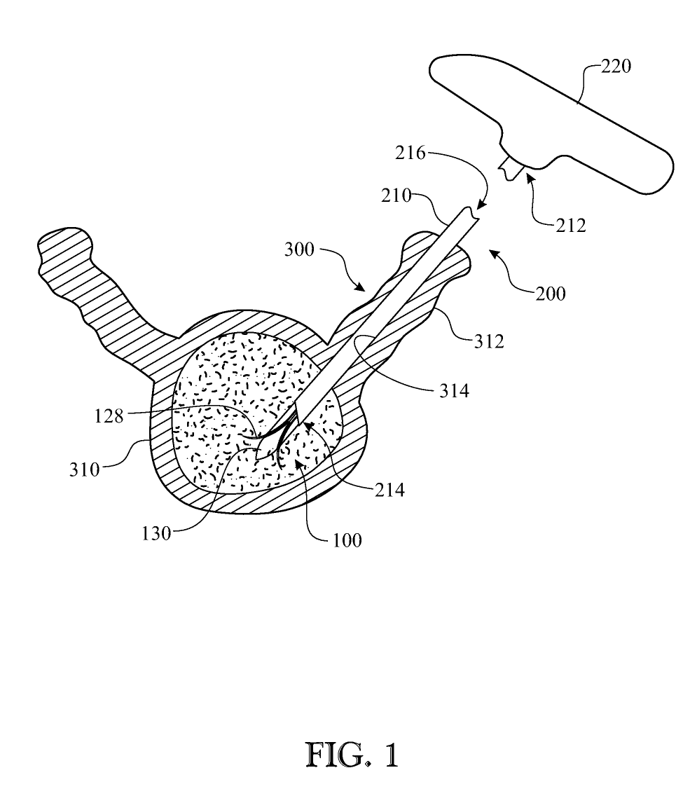

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. In other implementations, well-known features and methods have not been described in detail so as not to obscure the invention. For purposes of description herein, the terms “upper”, “lower”, “left”, “right”, “front”, “back”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments, which may be disclosed herein, are not to be considered as limiting, unless the claims expressly state otherwise.

Reference is now made to FIG. 1 where reference numeral 100 designates generally an anchor wire, guide wire usable in surgical procedures in combination with a surgical tool such as a Jamshidi needle 200, drill or tap (not shown), or a surgical device such as a screw, plate or implant. Guide wires (sometimes referred to as Kirschner wires) are well known in the art. Jamshidi needles are also well known in the art and have a shank 210 comprising a thru bore 216 (as would a drill, tap or screw) and a needle shank distal end 214. It is noted that the needle shank distal end 214 may include a tapered circumferential edge, a perpendicular or chamfered end, and any other known configuration. A handle 220 may also be provided at the needle proximal end 212 of the shank 210 for facilitating insertion of the shank 210 into a surgical site 300, such as a vertebra 310 with a pedicle 312 in a patient such as a human. A surgeon may manipulate the Jamshidi needle 200 using the handle 220, and may also apply impact force to the shank 210 by striking the handle with a hand or impact tool such as a hammer/mallet. Jamshidi needles 120 are used to penetrate bone in the performance of a surgical procedure such as attaching a screw (not shown) to bone. After forming a hole 314 with the Jamshidi needle 200, the anchoring guide wire assembly 100 is inserted into the interior of the bone and the Jamshidi needle 200 may be removed, leaving the anchoring guide wire assembly 100 in place. If care is not taken during surgery, the known guide wire may be pushed through the opposing bone wall creating a risk of injury. The present invention is a solution to this potential problem.

The anchoring guide wire assembly 100 is used as a pilot or guide for other surgical tools or devices such as drills, taps, plates, implants and screws. In the attachment of a screw, the screw typically has a through bore that receives the anchoring guide wire assembly 100 for guiding the screw to a drilled and tapped hole 314. After installation of the screw, the anchoring guide wire assembly 100 is then extracted from the through bore.

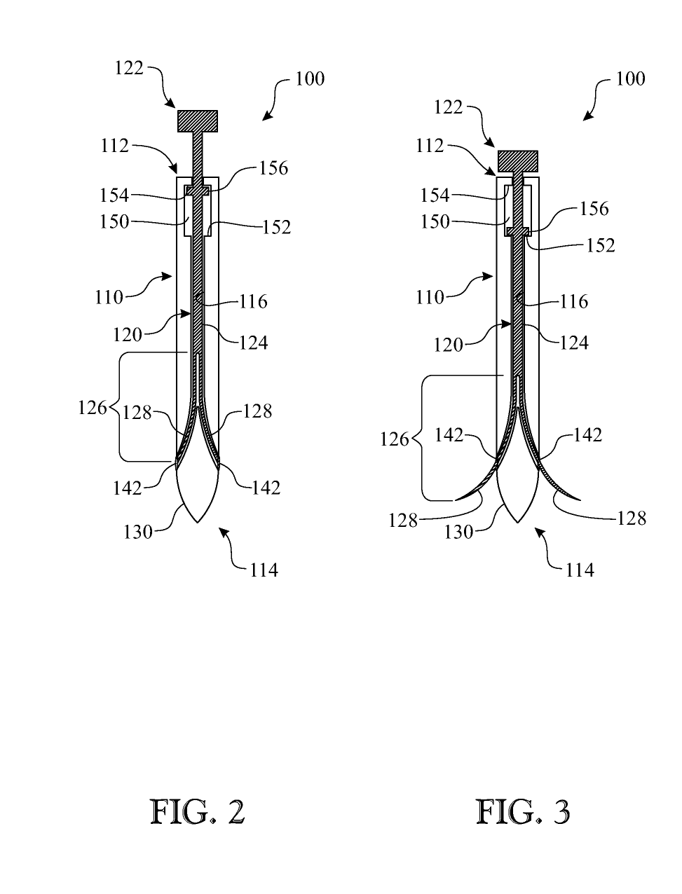

Reference is now made to FIGS. 2 and 3 in which the anchoring guide wire assembly 100 is an assembly comprising a guide wire hollowed sleeve member 110 and a guide wire location retention member 120 slideably assembly within a guide wire hollowed sleeve member 110 passing therethrough. The guide wire hollowed sleeve member 110 includes a guide wire penetrating head 130 located at a sleeve distal end 114 and access to a central aperture 116 initiating at an opposite or sleeve proximal end 112. It is understood that the exterior cross sectional shape of the anchoring guide wire assembly 100 may be formed having any cross sectional shape, including circular, elliptical, triangular, rectangular, octagonal, and the like. Similarly, it is understood that the interior cross sectional shape of the central aperture 116 and the exterior cross sectional shape of the respective guide wire location retention member 120 may be formed having any cross sectional shape, including circular, elliptical, triangular, rectangular, octagonal, and the like. The length of the anchoring guide wire assembly 100 is preferably long enough to extend beyond both ends of the surgical tool being used, e.g., a Jamshidi needle 200. The exterior, cross sectional size and shape of the anchoring guide wire assembly 100 is such to enable free sliding motion along the thru bore 216 of the Jamshidi needle 200. The guide wire penetrating head 130 is provided to facilitate penetration of the anchoring guide wire assembly 100, the Jamshidi needle 200, or both through a desired bone structure, cartilage, soft tissue, and the like of a patient. In the exemplary embodiment, the guide wire penetrating head 130 is integrated with a distal end 114 of the guide wire hollowed sleeve member 110. Other exemplary embodiments, presented later herein, will present alternate configurations, where the guide wire penetrating head is integrated onto a distal end of the guide wire location retention member.

In the exemplary embodiment, the guide wire hollowed sleeve member 110 is configured having a central aperture 116, which extends from the sleeve proximal end 112 and continues axially along a length of the guide wire hollowed sleeve member 110 terminating at bi-laterally exits referred to as retention member operational ports 142. The retention member operational ports 142 are provided through a sidewall of the guide wire hollowed sleeve member 110 at a location defining a proximal end of the guide wire penetrating head 130.

Guide wire location retention member 120 is slideably assembled within the central aperture 116 such that the guide wire location retention member 120 may slideably translate between a retracted state as shown in FIG. 2 and a deployed state shown in FIG. 3. The guide wire location retention member 120 is defined having a retention member proximal end 122 located at a first end of a retention member central section 124 and an retention member distal end 126 located at an opposite end of the retention member central section 124. In one embodiment, the retention member distal end 126 is configured having a bi-lateral pair of extending retention arms 128 which branch adjacent to retention member distal end 126 and extend through retention member operational ports 142 in the deployed state.

The exemplary embodiment includes an optional retention arm protruding distance control chamber 150 formed within the guide wire hollowed sleeve member 110 the distance control chamber 150 being located adjacent to the sleeve proximal end 112. The retention arm protruding distance control chamber 150 is designed having a size and configuration to provide a deployment range constraining feature for use with the guide wire location retention member 120. The retention arm protruding distance control chamber 150 includes a chamber extension limiting surface 152 and a chamber retraction limiting surface 154, wherein the chamber extension limiting surface 152 and the chamber retraction limiting surface 154 are located at longitudinally opposing ends of the retention arm protruding distance control chamber 150. Adjacent to the retention member proximal end 122 of guide wire location retention member 120 is a retention member motion limiting collar 156 which is sized and configured for translating movement within retention arm protruding distance control chamber 150. As shown in FIG. 2, the retention member motion-limiting collar 156 engages with the chamber retraction-limiting surface 154 and thereby ensures each extending retention arm 128 is retracted completely within the respective retention member operational port 142 for insertion and removal of the anchoring guide wire assembly 100 during use. As shown in FIG. 3, the retention member motion-limiting collar 156 engages with the chamber extension limiting surface 152 and thereby limits the maximum distance each extending retention arm 128 may laterally extend beyond the respective retention member operational port 142.

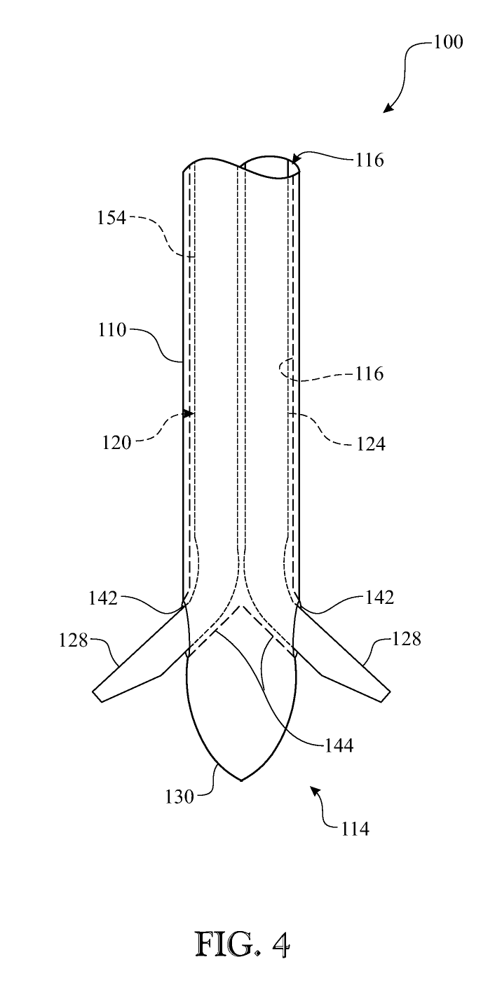

An enlarged view of extending retention arms 128 is illustrated in a deployed state as presented in FIG. 4. The extending retention arms 128 are deployed by slideably positioning the guide wire location retention member 120, causing the extending retention arms 128 to temporarily seat in position proximate the surgical site. As a result of seating the extending retention arms 128 in position, forward translation/movement of the sleeve distal end 114 is prevented and rearward movement or pullout is significantly limited. Guide wire penetrating head 130 includes a retention member diverter surface 144 for directed each extending retention arm 128 through each respective retention member operational port 142. The retention member diverter surface 144 facilitates the transition of the extending retention arm 128 from a longitudinally aligned retracted state into a laterally oriented deployed state.

The anchoring guide wire assembly 100 enables the medical professional to deploy the extending retention arms 128 when the anchor wire is positioned at a desire location. The anchoring guide wire assembly 100 additionally enables the medical professional the ability to reposition the anchoring guide wire assembly 100. The repositioning process would be accomplished by adjusting the tubular guide wire hollowed sleeve member 110 to retract the extending retention arms 128, reposition the anchoring guide wire assembly 100, then adjusting the tubular guide wire hollowed sleeve member 110 to deploy or redeploy the extending retention arms 128.

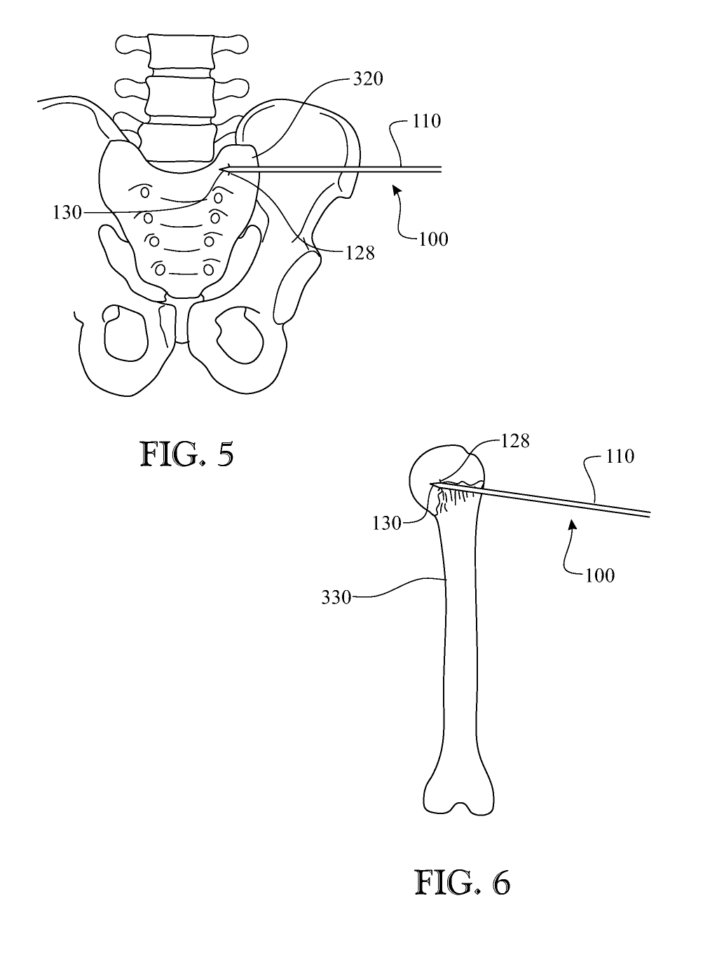

Reference is now made to FIGS. 5 and 6, which illustrate exemplary applications of the anchoring guide wire assembly 100 of the present invention in a deployed state within various exemplary bone structures of a patient. In FIG. 5, anchoring guide wire assembly 100 is temporarily anchored within a portion of a pelvic bone 320. The extending retention arms 128, in a deployed state, prevent any forward translation/movement of the sleeve distal end 114 and significantly limit any rearward movement or pullout. Similarly, in FIG. 6, anchoring guide wire assembly 100 is coupled to an upper portion of a humerus bone 330 such that extending retention arms 128 likewise prevent any additional forward movement and significantly limit any rearward movement or pullout of anchoring guide wire assembly 100. It is understood that the exemplary embodiments presented throughout this disclosure may be utilized in any medical procedure where a guide wire is employed.

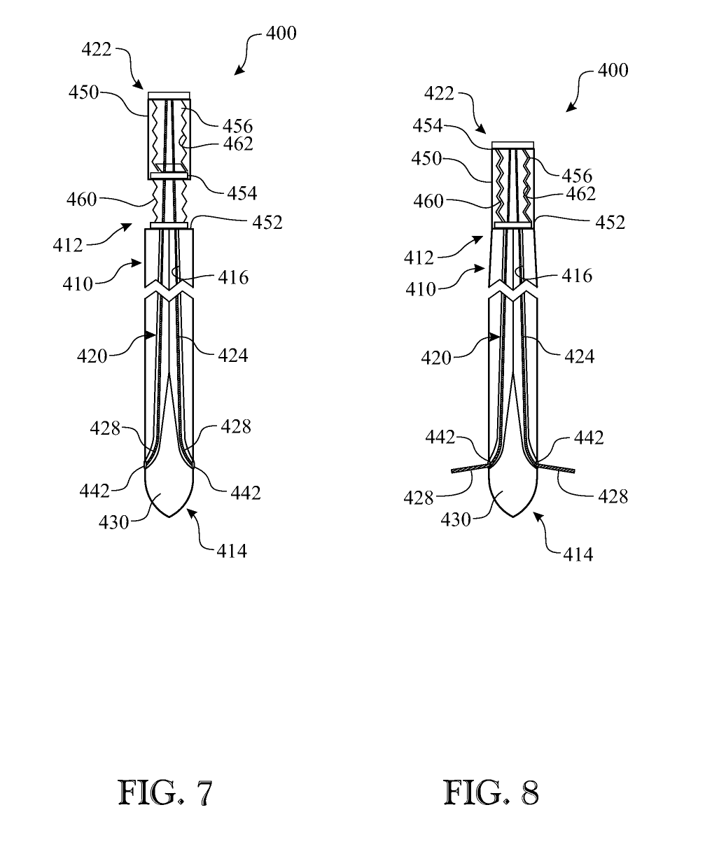

A anchoring guide wire assembly 400 is an enhanced version of the anchoring guide wire assembly 100, introducing an optional indexing configuration for controlling deployment of extending retention arms 428 of an anchoring guide wire assembly 400, as illustrated in FIGS. 7 and 8. Like features of anchoring guide wire assembly 400 and anchoring guide wire assembly 100 are numbered the same except preceded by the numeral ‘4’. Additionally, the guide wire location retention member 420 is fabricated where the pair of extending retention arms 428 extends partially or completely towards the retention member proximal end 422. Where the retention member central/shank section 124 is fabricated having a single segment; the retention member central section 424 can be fabricated having a single or multiple segments.

The extending retention arms 428 extend and retract through each respective retention member operational port 442. The projecting distance of the extending retention arm 428 can be controlled by the optional indexing configuration. The extending retention arms 428 are shown retracted within the guide wire hollowed sleeve member 410 in FIG. 7 and fully deployed in FIG. 8, where each extending retention arm 428 extends laterally outward from a retention member operational port 442 of the guide wire hollowed sleeve member 410. The exemplary indexing configuration includes a retention member indexing end cap 450 carried at a retention member proximal end 422 of the guide wire location retention member 420. The retention member indexing end cap 450 includes a cap indexing cavity 456 that is configured with a cavity interior indexing surface 462 that operatively couples with first indexing surface 460 of guide wire hollowed sleeve member 410. In this embodiment, a retention member proximal end 422 of guide wire location retention member 420 is coupled to an inner surface of retention member indexing end cap 450 such that when retention member indexing end cap 450 is moved, guide wire location retention member 420 correspondingly moves. It is contemplated that the indexing configuration may include one of many known mechanical designs that provide for measured/calibrated linear movement between a first and second member (i.e., guide wire hollowed sleeve member 410 and guide wire location retention member 420). The anchoring guide wire assembly 400 is an exemplary embodiment that enables the user to control and vary the distance and deployment of the extending retention arm 428.

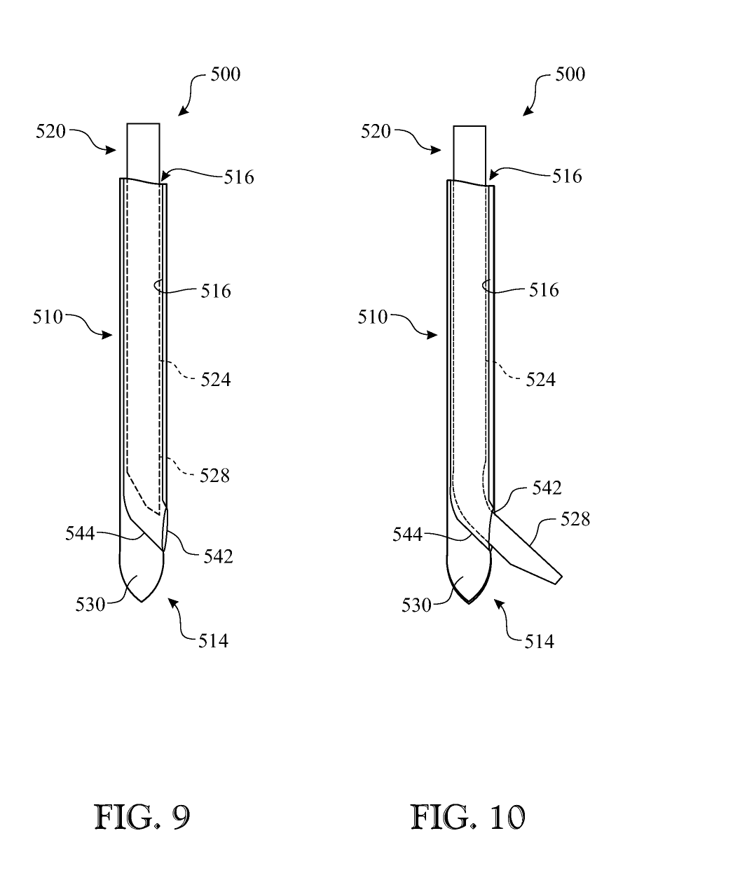

Attention is now directed to FIGS. 9 and 10, which illustrate another alternate exemplary embodiment of the present invention referenced as an anchoring guide wire assembly 500. The anchoring guide wire assembly 500 is similar to the anchoring guide wire assembly 100, while being modified towards applications requiring a guide wire having a smaller diameter. Like features of anchoring guide wire assembly 500 and anchoring guide wire assembly 100 are numbered the same except preceded by the numeral ‘5’. Similar to the anchoring guide wire assembly 100, a guide wire location retention member 520 is slideably assembled within a sleeve central aperture 516 of a guide wire hollowed sleeve member 510. Where the anchoring guide wire assembly 100 includes a pair of extending retention arms 128 and respective retention member operational ports 142, the anchoring guide wire assembly 500 includes a single extending retention arm 528 and a single respective retention member operational port 542. When the guide wire location retention member 520 is longitudinally positioned within the guide wire hollowed sleeve member 510, the extending retention arm 528 is retracted therein or deployed. During deployment, the extending retention arm 528 is directed towards and through the retention member operational port 542 by a retention member diverter surface 544. The extending retention arm 528 is shown retracted within the guide wire hollowed sleeve member 510 in FIG. 9 and fully deployed in FIG. 10, where the extending retention arm 528 extends laterally outward through the retention member operational port 542 of the guide wire hollowed sleeve member 510. Due to the singular configuration of guide wire location retention member 520, having a single extending retention arm 528, both the guide wire hollowed sleeve member 510 and respective guide wire location retention member 520 may be designed having a significantly smaller diameter. The anchoring guide wire assembly 500 of this alternate exemplary embodiment is contemplated to include applicable and similar features as discussed above with reference to FIGS. 1 through 8.

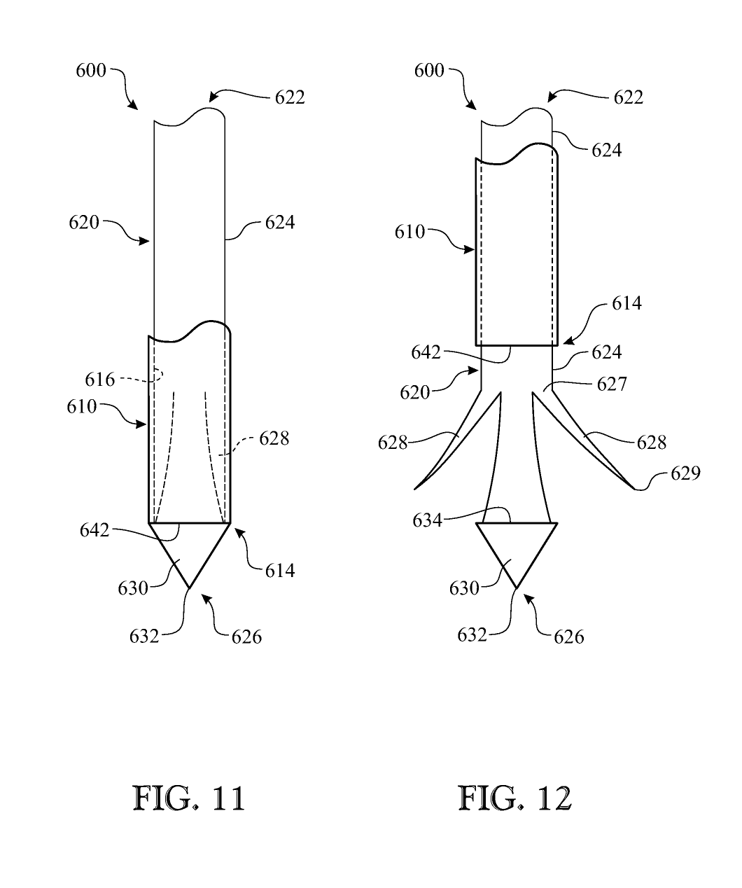

The previously presented embodiments of the anchoring guide wire assembly 100, 400, 500 integrated the guide wire penetrating head 130, 430, 530 into the sleeve distal end 114, 414, 514. Alternatively, the following exemplary embodiments integrate a guide wire assembly penetrating head onto a retention member distal end of a guide wire location retention member. A first exemplary embodiment of this alternative configuration is referred to as an anchoring guide wire assembly 600 and is presented in FIGS. 11 and 12. The anchoring guide wire assembly 600 is similar to the anchoring guide wire assembly 100, 400, 500, while being modified integrating a guide wire assembly penetrating head 630 upon a retention member distal end 626 of a guide wire location retention member 620. Like features of anchoring guide wire assembly 600 and anchoring guide wire assembly 100, 400, 500 are numbered the same except preceded by the numeral ‘6’.

Similar to the anchoring guide wire assembly 100, 400, 500, a guide wire location retention member 620 is slideably assembled within a sleeve central aperture 616 of a guide wire hollowed sleeve member 610. The guide wire hollowed sleeve member 610 is defined having a retention member operational port 642 at a sleeve distal end 614 thereof. A penetrating head trailing edge 634 is provided at a nominal distance from a penetrating tip 632 of the guide wire assembly penetrating head 630. The penetrating head trailing edge 634 preferably defines an upper end of the guide wire assembly penetrating head 630. At least one extending retention arm 628 is provided along the guide wire location retention member 620 at a location proximate the penetrating head trailing edge 634. The extending retention arm 628 are integrally formed within the guide wire location retention member 620, originating from the retention member central section 624 at a retention arm initiating end 627, and extending a length that terminates at a retention arm distal end 629. It is desired that the retention arm distal end 629 is located adjacent to the penetrating head trailing edge 634 when the at least one extending retention arm 628 is retracted within the sleeve central aperture 616 of the guide wire hollowed sleeve member 610.

The extending retention arms 628 are elastically deformed, having a natural deployed shape as presented in the deployed configuration illustrated in FIG. 12. The extending retention arms 628 are fully deployed when the retention member operational port 642 is longitudinally translated to a position adjacent to the retention arm's initiating end 627.

The extending retention arm 628 are retracted when the penetrating head trailing edge 634 and retention member operational port 642 are drawn together by slideably positioning the guide wire hollowed sleeve member 610 and the guide wire location retention member 620 accordingly. As the penetrating head trailing edge 634 and retention member operational port 642 are drawn together by the sliding motion between the guide wire hollowed sleeve member 610 and guide wire location retention member 620, the sleeve central aperture 616 elastically contracts each of the at least one extending retention arm 628 therein. The anchoring guide wire assembly 600 is prepared for insertion into a patient when the extending retention arms 628 are in a fully retracted configuration. The extending retention arms 628 are completely retracted when the penetrating head trailing edge 634 abuts the retention member operational port 642, as illustrated in FIG. 11.

The guide wire assembly penetrating head 630 is provided and configured to facilitate penetration/piercing of anatomical structures such as bone, cartilage, soft tissue, and the like. In one exemplary embodiment, the guide wire assembly penetrating head 630 may be configured as a trocar or three-sided point. It is understood that any suitable shape may be employed for the guide wire assembly penetrating head 630, including a threaded tip, a blunt tip, a sharp tip, a chisel tip, a pencil tip, and the like. It is desired that the maximum diameter of the guide wire assembly penetrating head 630 be substantially equal to the outside diameter of sleeve distal end 614.

It is contemplated, that anchoring guide wire assembly 600 may be selectively moved between a retracted state (shown in FIG. 11) and a deployed state (shown in FIG. 12) by longitudinally translating locking guide wire hollowed sleeve member 610 with respect to guide wire location retention member 620. When the guide wire assembly penetrating head 630 is positioned at a desired location, the guide wire hollowed sleeve member 610 is longitudinally translated away from the retention member distal end 626, causing the extending retention arm 628 to deploy. When deployed, the retention arm distal end 629 of each extending retention arm 628 is located beyond the outside diameter of guide wire hollowed sleeve member 610 and is oriented towards a penetrating tip 632 of the guide wire assembly penetrating head 630. As a result of seating the extending retention arms 628 in position, forward translation/movement of the guide wire location retention member 620 is prevented and rearward movement or pullout is significantly limited.

In an exemplary embodiment, the extending retention arm 628 may be spring loaded such that when the retention arm distal end 629 are longitudinally translated beyond the retention member operational port 642 of the guide wire hollowed sleeve member 610, the extending retention arm 628 laterally extend outwards. When the extending retention arms 628 are deployed, the retention arm distal end 629 adequate seat within the surrounding region, thus retaining the guide wire location retention member 620 in position. In another exemplary embodiment, the deflection of the extending retention arm 628 may be effectuated by fabricating the guide wire location retention member 620 out of a deformable material which will allows the retention member distal end 626 to be selectively configured between a retracted state (shown in FIG. 11) and a deployed state (shown in FIG. 12).

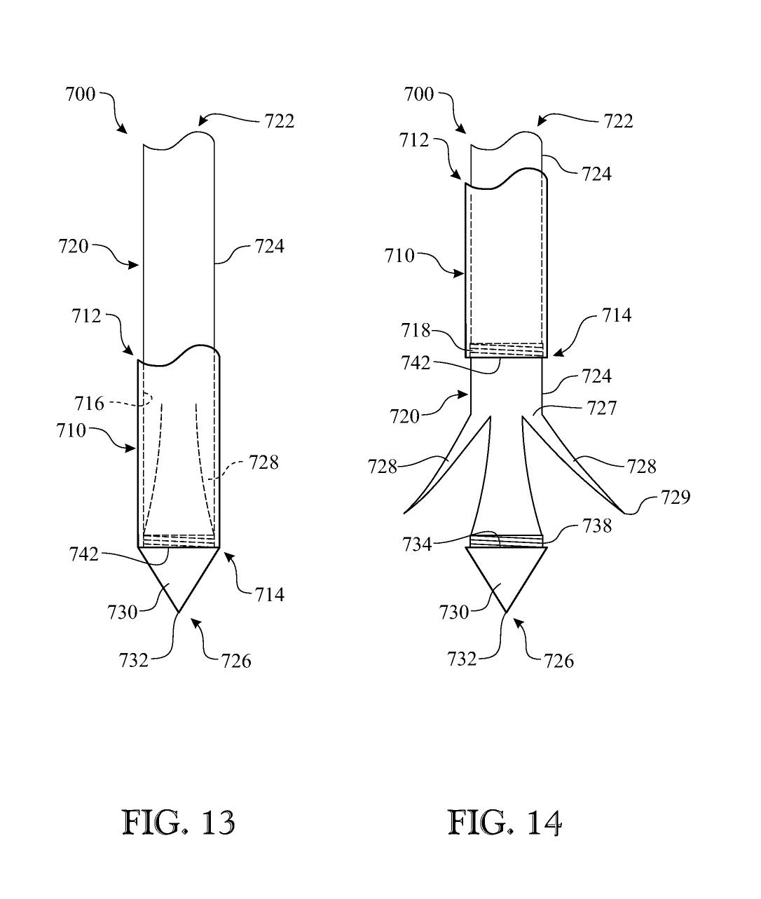

The anchoring guide wire assembly 600 can be modified with an inclusion of a temporary joining feature provided between the guide wire hollowed sleeve member and the guide wire location retention member. An exemplary embodiment of this optional feature is illustrated in an exemplary embodiment referred to as an anchoring guide wire assembly 700 and is presented in FIGS. 13 and 14. The exemplary anchoring guide wire assembly 700 includes like features of the anchoring guide wire assembly 600, which are numbered the same except preceded by the numeral ‘7’. It is further understood that the disclosed feature may be included on other embodiments having a guide wire assembly penetrating head 730 integrated into a retention member distal end 726 of a guide wire location retention member 720.

A first engaging feature, such as a sleeve female threading 718 may be integrated into a sleeve distal end 714 of the guide wire hollowed sleeve member 710. The first engaging feature would be located proximate the retention member operational port 742. The exemplary sleeve female threading 718 would be formed within an inner surface of a sleeve central aperture 716 initiating the sleeve distal end 714 and continue axially toward a sleeve proximal end 712 (shown cut) of the guide wire hollowed sleeve member 710. Correspondingly, a mating engaging feature, such as a penetrating head male threading 738 may be integrated into a 720. The mating engaging feature would be located proximate the penetrating head trailing edge 734. The exemplary penetrating head male threading 738 would initiate proximate a penetrating head trailing edge 734 and continue axially toward a retention member proximal end 722 of the guide wire location retention member 720. The sleeve female threading 718 and penetrating head male threading 738 would have mating thread size and pitch allowing for removable engagement. The guide wire hollowed sleeve member 710 would be temporarily secured to the guide wire location retention member 720 by rotationally engaging the sleeve female threading 718 and penetrating head male threading 738, thus ensuring preservation of the anchoring guide wire assembly 700 in a retracted configuration until desired. The surgical member would then rotate the guide wire hollowed sleeve member 710 or guide wire location retention member 720 to disengage the sleeve female threading 718 and penetrating head male threading 738, allowing separation between the retention member operational port 742 and the penetrating head trailing edge 734 to deploy the extending retention arms 728. It is understood that the sleeve female threading 718 and penetrating head male threading 738 are only exemplary and any engaging feature interface configuration may be employed therein. The same is true for the proximal end.

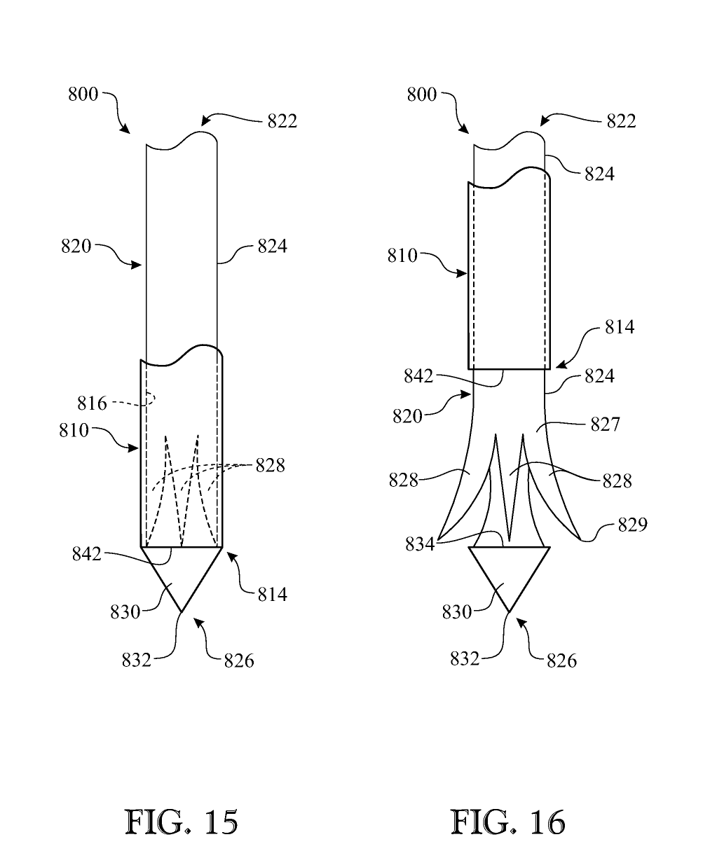

The anchoring guide wire assembly 600 included a pair of equally spaced extending retention arms 628. an anchoring guide wire assembly 800 is similar to the anchoring guide wire assembly 600, modified to include four (4) equally spaced extending retention arms 828, as illustrated in FIGS. 15 and 16. The anchoring guide wire assembly 800 features and functions are similar to the anchoring guide wire assembly 600, with a retracted configuration being illustrated in FIG. 15 and a deployed configuration being illustrated in FIG. 16. Like features of anchoring guide wire assembly 800 and anchoring guide wire assembly 600 are numbered the same except preceded by the numeral ‘8’. The extending retention arms 828 are deployed by slideably positioning the tubular guide wire hollowed sleeve member 810, causing the extending retention arms 828 to temporarily seat in position proximate the surgical site. As a result of seating the extending retention arms 828 in position, forward translation/movement of the sleeve distal end 814 is prevented and rearward movement or pullout is significantly limited. The anchoring guide wire assembly 800 additionally enables the medical professional the ability to reposition the anchoring guide wire assembly 800. The repositioning process would be accomplished by adjusting the tubular guide wire hollowed sleeve member 810 to retract the extending retention arms 828, reposition the anchoring guide wire assembly 800, then adjusting the tubular guide wire hollowed sleeve member 810 to deploy or redeploy the extending retention arms 828.

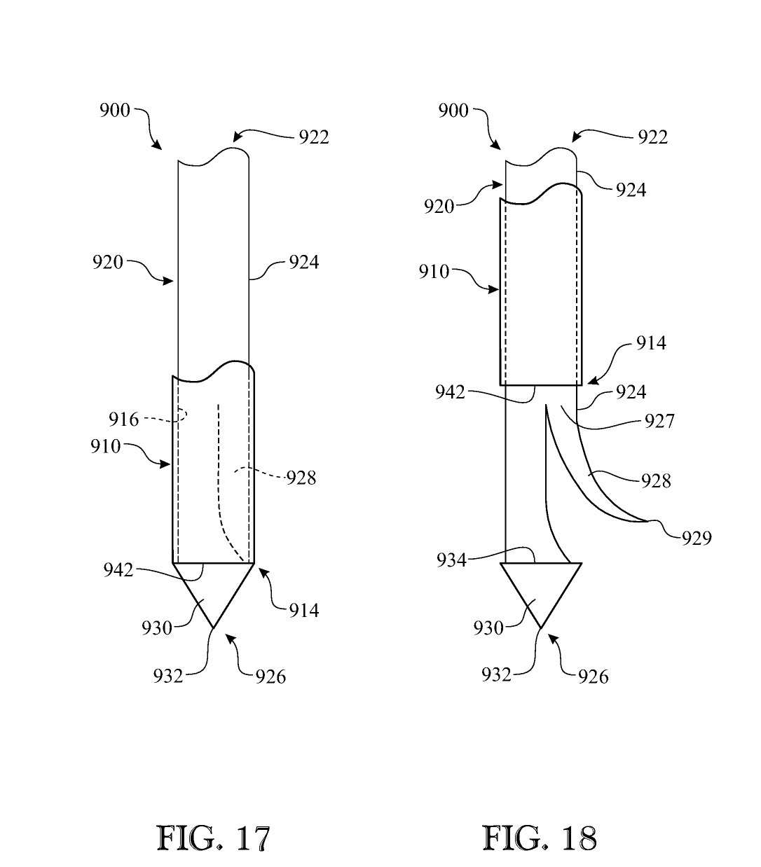

The anchoring guide wire assembly 500 and anchoring guide wire assembly 600 are combined in an exemplary embodiment referred to as an anchoring guide wire assembly 900 and illustrated in FIGS. 17 and 18. Like features of anchoring guide wire assembly 900 and anchoring guide wire assembly 500, 600 are numbered the same except preceded by the numeral ‘9’. The anchoring guide wire assembly 900 includes a guide wire assembly penetrating head 930 disposed at a retention member distal end 926 of a guide wire location retention member 920, similar to the anchoring guide wire assembly 600. The anchoring guide wire assembly 900 includes a single extending retention arm 928 similar to the anchoring guide wire assembly 500, providing for a guide wire having a reduced overall diameter. The anchoring guide wire assembly 900 functions similarly to any of the anchoring guide wire assembly 600, 700, 800, with a retracted configuration being illustrated in FIG. 17 and a deployed configuration being illustrated in FIG. 18.

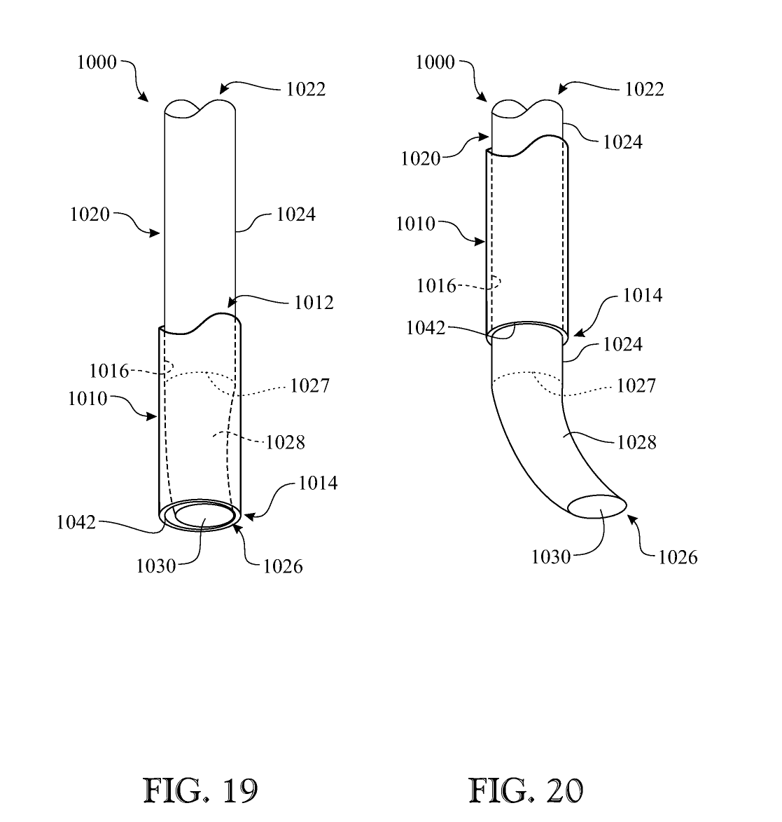

The anchoring guide wire assembly 500 is modified to form an exemplary embodiment referred to as an anchoring guide wire assembly 1000 and illustrated in FIGS. 19 and 20. Like features of anchoring guide wire assembly 1000 and anchoring guide wire assembly 500, 600 are numbered the same except preceded by the numeral ‘10’. The anchoring guide wire assembly 1000 includes a guide wire assembly penetrating head 1030 disposed at a retention member distal end 1026 of a guide wire location retention member 1020, similar to the anchoring guide wire assembly 600. The anchoring guide wire assembly 1000 includes an extending retention arm 1028 similar to the anchoring guide wire assembly 500, where the extending retention arm 1028 includes a natural bend outward from a longitudinal axis of the retention member central section 1024. The anchoring guide wire assembly 1000 functions similarly to any of the anchoring guide wire assembly 600, 700, 800, 900 with a retracted configuration being illustrated in FIG. 19 and a deployed configuration being illustrated in FIG. 20. During deployment, the natural curvature of the extending retention arm 1028 causes the extending retention arm 1028 to bend outward from the longitudinal axis of the retention member central section 1024 as the guide wire hollowed sleeve member 1010 is retracted, exposing the guide wire location retention member 1020.

One of ordinary skill in the art would readily appreciate that various numbers/configurations of retention members are possible. All the permutations are not discussed so as not to obscure the invention herein. It is understood that the anchoring guide wire assembly 100, 400, 500, 600, 700, 800, 900 can include any number of spatially arranged retention members 128, 428, 528, 628, 728, 828, 928, wherein the spacing between adjacent retention members 128, 428, 528, 628, 728, 828, 928 may be alike or varied.

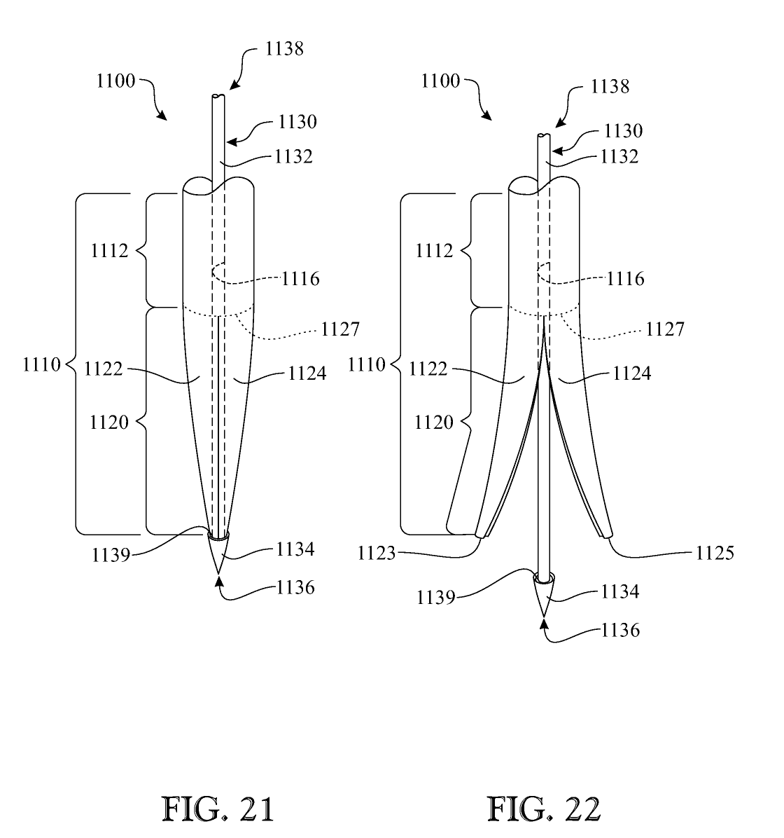

It is understood that the concept can be reversed, as illustrated by the exemplary embodiment of an anchoring guide wire assembly 1100 presented in FIGS. 21 and 22. The guide wire hollowed sleeve member 1110 can be defined by two segments; a sleeve tubular segment 1112 and a sleeve distal retention section 1120. The sleeve tubular segment 1112 is preferably a continuous tubular section having a sleeve central aperture 1116 passing therethrough. The sleeve distal retention section 1120 is integrated into a distal end of the sleeve tubular segment 1112. The sleeve distal retention section 1120 includes one or more retention arms 1122, 1124. Each retention arm 1122, 1124 is pre-bent where a retention arm distal end 1123, 1125 is located at a distance from a longitudinal axis of the sleeve central aperture 1116 as a result of the pre-bent shape of the respective retention arm 1122, 1124. The guide wire hollowed sleeve member 1110 can be fabricated using a tubular shaped member, such as the sleeve tubular segment 1112 and creating one or more slits extending inward from the distal end 1123, 1125. The slit would terminate at a retention segment transition 1127. The ends would be formed, bending each retention arm distal end 1123, 1125 outward from the longitudinal axis of the sleeve central aperture 1116. The bending would create a cantilever spring force into the first retention arm 1122 and second retention arm 1124, where the natural shape of each retention arm 1122, 1124 would place the each retention arm distal end 1123, 1125 at a distance from the sleeve central aperture 1116 and a retracted, insertion configuration of each retention arm 1122, 1124 would place the each retention arm distal end 1123, 1125 proximate the sleeve central aperture 1116.

A central actuating member 1130 passes through the sleeve central aperture 1116. The central actuating member 1130 is fabricated having a penetrating head 1134 disposed upon a distal end of the actuating member shank 1132. The penetrating head 1134 is shaped to include a pointed distal end 1136 formed at a distal end and a retention arm distal end retention feature 1139 formed at an attachment end. The anchoring guide wire assembly 1100 is placed into an installation configuration by contracting the bent first retention arm 1122 and second retention arm 1124, where the first retention arm distal end 1123 and second retention arm distal end 1125 are contracted and placed within the retention arm distal end retention feature 1139 of the penetrating head 1134. The retention arm distal end retention feature 1139 can be formed as a recess within the attachment end of the penetrating head 1134.

In use, initially, the anchoring guide wire assembly 1100 would be an insertion configuration, where the retention arm distal ends 1123, 1125 are retained in a retracted position by the retention arm distal end retention feature 1139. The surgeon or surgical assistant would insert the anchoring guide wire assembly 1100 into the desired location. The pointed distal end 1136 would engage with the target location of the patient. Once located, the surgeon or surgical assistant would slightly withdraw the guide wire hollowed sleeve member 1110, causing retention arms 1122, 1124 to deploy. Alternatively, the anchoring guide wire assembly 1100 would be properly located and the central actuating member 1130 would be driven slightly forward from the guide wire hollowed sleeve member 1110, causing the retention arm distal end retention feature 1139 and the retention arm distal ends 1123, 1125 to separate, resulting in the deployment of the retention arms 1122, 1124. It is noted that the operation of the anchoring guide wire assembly 1100 is accomplished by gripping the proximal ends 1118, 1138 of the guide wire hollowed sleeve member 1110 and the central actuating member 1130 respectively.

It is understood that any of the deployment range constraining configurations described herein can be integrated into any of the disclosed embodiments or variations thereof. It is further noted that any deployment range constraining configurations known by those skilled in the art may be employed.

It is further understood that the guide wire embodiments disclosed herein may include various designs for mechanically coupling the proximal end with the proximal end/handle of the Jamshidi needle. This mechanical coupling may be implemented by use of a removable collar, locking screw cap, set screws or other mechanical couplers that provide releasable coupling between two mechanical elements.

The present invention also includes a method of conducting a medical procedure using an anchoring guide wire assembly 100, as illustrated in FIG. 2. A surgeon or other medical personnel places a surgical tool end at the surgical site. The initial surgical tool used preferably has a guiding through bore opening at the distal tool end such as that described above for a Jamshidi needle 200. The anchoring guide wire assembly 100 is guided to the site by passing through the thru bore 216 until the guide wire penetrating head 130 of the sleeve distal end 114 of the anchoring guide wire assembly 100 is positioned at a desired location. The combination of the anchoring guide wire assembly 100 and Jamshidi needle 200 are then appropriately positioned within a bone structure, cartilage, soft tissue, and the like, as required by the particular medical procedure. After accurately locating the guide wire penetrating head 130 of the anchoring guide wire assembly 100, the Jamshidi needle 200 may be partial drawn back or conversely the anchoring guide wire assembly 100 may be slightly advanced beyond the needle shank distal end 214 of the Jamshidi needle 200 so that the retention member operational port 142 are exposed. The guide wire location retention member 120 is then positioned into a deployed state (see FIG. 3) such that extending retention arms 128 extend laterally outward from the sleeve distal end 114 of anchoring guide wire assembly 100. In the deployed state, anchoring guide wire assembly 100 is prevented from further forward movement or additional penetration of the bone structure, cartilage, soft tissue, and the like; and rearward movement or pullout is significantly limited. Once the anchoring guide wire assembly 100 is securely coupled within the desired bone structure, cartilage, soft tissue, and the like, the Jamshidi needle 200 may be withdrawn leaving the anchoring guide wire assembly 100 in place. The anchoring guide wire assembly 100 may now be used by the surgeon to guide various tools, devices, components, or combinations thereof, to the surgical site. Subsequent to installing any required components into the bone structure, cartilage, soft tissue, and the like, the anchoring guide wire assembly 100 may be removed or repositioned by positioning the guide wire location retention member 120 into a retracted state as illustrated in FIG. 2.

The alternate embodiments disclosed herein may be similarly used in accordance with the method described above. In these alternate embodiments, the actuation of the locking member from a retracted state into a deployed state may vary depending upon the actual embodiment being used. For example, in an embodiment as illustrated in FIGS. 13 and 14 there are additional steps required to disengage and reengage the mating threads. Similarly, in an embodiment where the extending retention arms are deployed by use of a thermally deformable material, there would need to be additional steps to effectuate the deployment thereof.

As will be now apparent to those skilled in the art, anchoring guide wire systems fabricated according to the teachings of the present invention are capable of substantially facilitating the placement of a guide wire during a surgical procedure. Since the present invention prevents the undesired translation of the anchor wire the device enhances and facilitates the medical treatment of the patient. In addition, the invention provides a device and method of use that greatly reduces the possibility of unintended patient injury. Importantly, the present invention provides an anchoring guide wire that permits accurate placement of the wire while simultaneously retaining the wire in place. Further, the anchoring guide wire enables the removal and repositioning of the anchoring guide wire as desired. Since the device includes a solid tip, the invention can be installed independent or in conjunction with any standard installation instrument for preparing and employing the device.

Although the above provides a full and complete disclosure of the preferred embodiments of the invention, various modifications, combinations, alternate constructions and equivalents will occur to those skilled in the art. For example, although the invention has been described with reference to constraining the movement of the locking member by employing a limiting collar or indexing component other components may be used to controllably translate the locking member with respect to the anchor wire. In addition, although the locking member has been described with extending retention arms oriented towards the penetrating point, other configurations are possible such as extending angularly or co-axially therefrom. It is intended that all matters in the foregoing description and shown in the accompanying drawings be interpreted as illustrative and not in a limiting sense. Therefore the above should not be construed as limiting the invention, which is defined by the appended claims and their legal equivalence.

What is claimed is:

1. An anchoring guide wire assembly for use in a surgical procedure, the anchoring guide wire assembly comprising:
a guide wire hollowed sleeve member having a guide wire hollowed sleeve member distal end located at a first end and a guide wire hollowed sleeve member proximal end located at an opposite end, at least one retention member operational port formed adjacent to a guide wire penetrating head formed on said guide wire distal end, a central aperture formed extending longitudinally through said guide wire hollowed sleeve member between said guide wire hollowed sleeve member proximal end and said at least one retention member operational port, and a solid penetrating head formed at said guide wire hollowed sleeve member distal end; and
a guide wire location retention member having a retention member distal end, a retention member proximal end and a retention member shank section spanning therebetween, said distal end configured with at least one extending retention arm,
wherein said guide wire location retention member is slideably assembled through said central aperture, said at least one extending retention arm is configured to extend through said at least one retention member operational port such that when said at least one extending retention arm is in a deployed configuration, said retention member extends laterally outward from said distal end of said guide wire hollowed sleeve member.

2. An anchoring guide wire assembly for use in a surgical procedure as recited in claim 1, said anchoring guide wire assembly being fabricated of medical grade materials, sterilized, and packaged to retain said sterility.

3. An anchoring guide wire assembly for use in a surgical procedure as recited in claim 1, the guide wire hollowed sleeve member further comprising a single retention member operational port; and
the guide wire location retention member comprising a single extending retention arm formed to bend laterally from a longitudinal axis of said guide wire location retention member.

4. An anchoring guide wire assembly for use in a surgical procedure as recited in claim 1, the guide wire hollowed sleeve member further comprising two retention member operational ports; and
the guide wire location retention member comprising a slit extending longitudinally from said retention member distal end forming two extending retention arms.

5. An anchoring guide wire assembly for use in a surgical procedure as recited in claim 1, the guide wire hollowed sleeve member further comprising two retention member operational ports; and
the guide wire location retention member comprising two extending retention arms joined together at a location proximate said guide wire location retention member proximal end.

6. An anchoring guide wire assembly for use in a surgical procedure as recited in claim 1, said guide wire hollowed sleeve member further comprising an indexing member located at said guide wire hollowed sleeve member proximal end and said guide wire location retention member comprising a mating indexing member located at said guide wire location retention member proximal end, wherein said indexing member and said mating indexing member engage to retain a positional relation between said guide wire hollowed sleeve member and said guide wire location retention member.

7. An anchoring guide wire assembly for use in a surgical procedure, the anchoring guide wire assembly comprising, in combination:
a guide wire hollowed sleeve member having a guide wire hollowed sleeve member proximal end located at a first end and a guide wire hollowed sleeve member distal end located at an opposite end, a central aperture formed extending longitudinally through said guide wire hollowed sleeve member between said guide wire hollowed sleeve member proximal end and said guide wire hollowed sleeve member distal end, and a retention member operational port formed at said guide wire hollowed sleeve member distal end; and
a guide wire location retention member having a retention member proximal end, a retention member distal end and a retention member shank section spanning therebetween, a solid guide wire assembly penetrating head carried at said retention member distal end, and at least one extending retention arm formed to extend laterally from said retention member shank section when placed into a natural state,
wherein said guide wire location retention member is slideably assembled through said retention member operational port, said retention member is retracted within said central aperture when said anchoring guide wire assembly is placed into a retracted, installing configuration and said retention member is extended laterally outward from said distal end of said guide wire hollowed sleeve member when said anchoring guide wire assembly is placed into a deployed configuration.

8. An anchoring guide wire assembly for use in a surgical procedure as recited in claim 7, said anchoring guide wire assembly being fabricated of medical grade materials, sterilized, and packaged to retain said sterility.

9. An anchoring guide wire assembly for use in a surgical procedure as recited in claim 7, said guide wire location retention member comprising a single extending retention arm formed to bend laterally from a longitudinal axis of said guide wire location retention member.

10. An anchoring guide wire assembly for use in a surgical procedure as recited in claim 7, said guide wire location retention member comprising at least two extending retention arms, each extending retention arm being formed to bend laterally from a longitudinal axis of said guide wire location retention member.

11. An anchoring guide wire assembly for use in a surgical procedure as recited in claim 7, said solid guide wire assembly penetrating head further comprising a penetrating head trailing edge having a diameter equal to an exterior diameter of said guide wire hollowed sleeve member and a sleeve engaging surface extending inward from said penetrating head trailing edge diameter, wherein said sleeve engaging surface engages with said guide wire hollowed sleeve distal end.

12. An anchoring guide wire assembly for use in a surgical procedure as recited in claim 7, said guide wire hollowed sleeve member further comprising an indexing member located at said guide wire hollowed sleeve member proximal end and said guide wire location retention member comprising a mating indexing member located at said guide wire location retention member proximal end, wherein said indexing member and said mating indexing member engage to retain a positional relation between said guide wire hollowed sleeve member and said guide wire location retention member.

13. An anchoring guide wire assembly for use in a surgical procedure as recited in claim 7, said guide wire hollowed sleeve member further comprising an indexing member located at said guide wire hollowed sleeve member distal end and said guide wire location retention member comprising a mating indexing member located at said guide wire location retention member distal end, wherein said indexing member and said mating indexing member engage to retain a positional relation between said guide wire hollowed sleeve member and said guide wire location retention member.

14. An anchoring guide wire assembly for use in a surgical procedure as recited in claim 13, said indexing member and said mating indexing member are designed to retain said guide wire location retention member in an installation configuration.

15. An anchoring guide wire assembly for use in a surgical procedure, the anchoring guide wire assembly comprising, in combination:
a guide wire hollowed sleeve member comprising:
a sleeve tubular segment,
a sleeve distal retention section comprising at least one extending retention arm, wherein each of said at least one extending retention arm is formed to extend laterally from a longitudinal axis of said sleeve tubular segment when placed into a natural state, each of said at least one extending retention arm having a retention arm distal end, and
a sleeve central aperture extending longitudinally through said sleeve tubular segment;
a central actuating member comprising:
an actuating member shaft having first end defined as an actuating member shaft proximal end and a second, opposite end defined as an actuating member shaft distal end, and
a solid penetrating head carried by said actuating member shaft distal end, said solid penetrating head shaped to include a retention arm distal end retention feature, wherein said retention arm distal end retention feature is defined to releasably engage with each of said retention arm distal end to temporarily retain said each of said at least one extending retention arm in a retracted configuration;
wherein said central actuating member is slideably assembled through said sleeve central aperture, said retention member is retracted within said central aperture when said anchoring guide wire assembly is placed into a retracted and each of said retention arm distal ends is retained in a retracted configuration by said retention arm distal end retention feature when said anchoring guide wire assembly is placed into an insertion configuration and said each of said retention arm distal ends is released from said retention arm distal end retention feature, enabling each of said at least one first retention arm to deploy when said anchoring guide wire assembly is placed into a deployed configuration.

16. An anchoring guide wire assembly for use in a surgical procedure as recited in claim 15, said anchoring guide wire assembly being fabricated of medical grade materials, sterilized, and packaged to retain said sterility.

17. An anchoring guide wire assembly for use in a surgical procedure as recited in claim 15, wherein said retention arm distal end retention feature is formed as a recess about a proximal end of said penetrating head.

18. An anchoring guide wire assembly for use in a surgical procedure as recited in claim 15, said guide wire hollowed sleeve member comprising at least two extending retention arms, each extending retention arm being formed to bend laterally from a longitudinal axis of said guide wire hollowed sleeve member.

19. An anchoring guide wire assembly for use in a surgical procedure as recited in claim 15, said sleeve distal retention section is formed in a tapering shape, wherein a diameter of said sleeve distal retention section continuously reduces between a retention segment transition and respective retention arm distal ends.
